(19) United States
(12) Patent Application Publication
(10) Pub. No.: US 2007/0270462 A1
(43) Pub. Date: Nov. 22, 2007

(54) 8-hydroxyquinoline compounds for treating a polyglutamine (polyQ)-expansion disease

(75) Inventors: Stephen Massa, San Francisco, CA (US); Trent Nguyen, Richmond, CA (US)

Correspondence Address: PAUL, HASTINGS, JANOFSKY & WALKER LLP, P.O. BOX 919092, SAN DIEGO, CA, 92191-9092, US

(73) Assignee: The Regents of the University of California, Oakland, CA (US)

(21) Appl. No.: 11/485,596

(22) Filed: Jul. 11, 2006

Related U.S. Application Data
(60) Provisional application No. 60/698,514, filed on Jul. 11, 2005.

(51) Int. Cl. A61K 31/47; A61P 21/00; A61P 43/00

(52) U.S. Cl. 514/311

(57) ABSTRACT

The present invention provides compositions and methods of treating a polyglutamine (poly Q) expansion disease in a subject using an 8-hydroxyquinoline compound.

### CROSS-REFERENCES TO RELATED APPLICATIONS

This application claims the benefit of U.S. patent application Ser. 60/698,514, filed Jul. 11, 2005, which is herein incorporated by reference in its entirety for all purposes.

### INCORPORATION OF SEQUENCE LISTING

A paper copy of the Sequence Listing and a computer readable form of the sequence listing on compact disk, containing the file named UCSF—SEQUENCE LISTING for Utility filing.doc, which is 35 KB in size (measured in MS-DOS), and which was created on Jul. 11, 2006, are herein incorporated by reference.

### BACKGROUND OF THE INVENTION

Huntington's disease (HD) is an autosomal dominant neurological disorder that causes progressive cognitive, motor, and psychiatric dysfunction over a 10 to 20 year disease course leading to death. (Bates et al, Huntington's Disease (Oxford University Press, Oxford), 2002; Young A B, J Clin Invest 111:299-302, 2003). HD is caused by CAG-repeat expansion (Snell et al, Nat Genet. 4:393-397, 1993) in the 5′ region of the IT15 gene that encodes the ubiquitously expressed 350-kDa protein huntingtin (Htt). The expanded CAG region encodes polyglutamine (polyQ) and hence HD is an example of a polyglutamine- or polyQ-expansion disease. When the polyQ region is 40 residues or more, there is virtually 100% penetrance of the disease phenotype (Ambrose et al, Somat Cell Mol Genet. 20:27-38, 1994). The physiologic roles of Htt are not fully understood, however, it is known to be important for vesicular transport of BDNF in axons (Gauthier et al, Cell 118:127-138, 2004).

The toxicity of polyQ-Htt appears to involve a gain-of-function (Schilling et al, Hum Mol Genet. 8:397-407, 1999) mechanism with possible adjunct involvement of impairment of physiologic Htt function (Gunawardena et al, Neuron 40:25-40, 2003). Studies suggest that diffusely distributed, mono- or oligomeric Htt is the predominant toxic form, and not the highly aggregated forms present in inclusion bodies (Arrasate et al, Nature 431:805-810, 2004; Watase et al, Neuron 34:905-919, 2002). New toxic activities may occur because the region containing the expanded polyglutamine tract binds to itself and other polyglutamine containing proteins, especially when cleaved from the full-length protein (Gafni et al, J Biol Chem 279:20211-20220, 2004), and may sequester and deactivate transcription factors such as TBP (Schaffar et al, Mol Cell 15:95-105, 2004), CBP (Jiang et al, Hum Mol Genet. 12:1-12, 2003), Sp1 and TAFII130 (Dunah et al, Science 296:2238-2243, 2002). Mutant Htt is also implicated in mitochondrial function defects, including initiation of the mitochondrial permeability transition (Choo et al, Hum Mol Genet. 13:1407-1420, 2004; Ruan et al, Hum Mol Genet. 13:669-681, 2004), and loss of trophic support due to decreased production (Zuccato et al, Nat Genet. 35:76-83, 2003; Zuccato et al, Science 293:493-398, 2001) and dominant-negative inhibition of transport (Gauthier et al, 2004 Supra) of BDNF. However, the relative contributions to pathogenesis of the many potentially toxic effects of polyglutamine expansion of Htt have yet to be determined.

Ongoing synthesis of mutant protein appears to be necessary to drive the pathogenic process. Yamamoto et al (Cell 101:57-66, 2000), using a tet-regulatable transgenic system, found that when expression of polyQ-htt exon 1 was inhibited in symptomatic mice, the neuropathologic and behavioral changes were reversed. Similar results have recently been reported in conditional model of SCAI (Xia et al, Nat Med 10:816-820, 2004). Finally, specific RNAi-mediated inhibition of mutant protein expression reverses symptoms and pathology in animal models of HD (Staber et al, Huntingtin Gene Silencing by Lentivirus-Delivered SHRNA Leads to Phenotypic Improvement in an HD Mouse Model Program No. 1015.2, 2004, Abstract Viewer/Itinerary Planner, Washington, D.C. Society for Nueroscience, 2004) and SCA (Xia et al, 2004 Supra). These studies suggest that interruption of mutant protein synthesis can abrogate, and even reverse, the pathologic process. However, the practical long-term clinical application of RNAi and other nucleotide-based technologies specifically targeting mutant protein production will require resolution of several problems, including blood-brain barrier penetration and immune responses.

Several small compounds targeting features of polyQ protein metabolism and toxic mechanisms noted above have been found to mitigate symptoms and/or pathology in the HD model animals (reviewed in Beal M F & Ferrante, Nat Rev Neurosci 5:373-384, 2004) and other compounds have been discovered in screens in in vitro polyQ-expansion disease models and inhibit aggregate formation or cell death (Heiser et al, Proc Natl Acad Sci USA 99(4): 16400-16406, 2002; Pollitt et al, Neuron 40:685-694, 2003). To date, however, there has been no report of a small molecule that can selectively disrupt expanded CAG-repeat expression.

Clioquinol (5-chloro-7-iodo-8-hydroxy-quinoline; CQ) is a Cu⁺⁺—Zn⁺⁺-binding 8-hydroxyquinoline compound which is capable of penetrating cells and diminish the production of reactive oxygen species (ROS) production. In addition, CQ can cross the blood-brain barrier, enhance Aβ aggregate dissolution, decrease Aβ toxicity, and be given safely under conditions of controlled dosing and vitamin supplementation. A phase II clinical trial showed early positive effects on Alzheimer's disease and no significant CQ toxicity (Ritchie et al, Arch Neurol 60:1685-1691, 2003).

Thus, there is a need in the art for new treatments of polyglutamine (polyQ) expansion diseases, such as Huntington's disease. The present invention meets these and other needs in the art.

### BRIEF SUMMARY OF THE INVENTION

It has been discovered that, surprisingly, 8-hydroxyquinoline compounds are useful in the treatment of polyglutamine (polyQ) expansion diseases, such as Huntington's disease.

In one aspect, the present invention provides methods of treating a polyglutamine (poly Q) expansion disease in a subject in need of such treatment. The method includes administering to the subject an effective amount of an 8-hydroxyquinoline compound.

Another aspect of the present invention is directed to a composition comprising an 8-hydroxyquinoline compound (e.g. CQ) and at least one other agent selected from suberoylanalide hydroxamic acid (SAHA), sodium butyrate, 2VAD-fmk, minocycline, creatine, coenzyme Q₁₀, riluzole, congo red and trehalose, said composition further comprising one or more pharmaceutically acceptable carriers and/or diluents.

### BRIEF DESCRIPTION OF THE DRAWINGS

FIG. 1 is a photographic and graphical representation showing that CQ selectively down-regulates expanded polyglutamine-egfp fluorescence in vitro. Bars and error bars represent mean+s.e.m. n≧3 for each data set. Fluorescence microscopy and quantitation of EGFP, htt^{exon1}−Q²⁵-egfp and htt^{exon1}−Q¹⁰³-egfp expressing cells. Scale bar, 100 μm. **P<0.03.

FIG. 2 is a graphical representation showing that CQ selectively down-regulates polyglutamine protein expression and decreases cell death in vitro. Bars and error bars represent mean+s.e.m. n≧3 for each data set. (a) Western blotting and quantiation of EGFP (GFP Ab) and htt^{exon1}ΓQ¹⁰³-egfp (polyQ Ab) cell lysates. ***P=0.02. (b) Viability (PI staining) of htt^{exon1}−Q¹⁰³-egfp cells treated with CQ. *P<0.03, ** P<0.002 versus vehicle control.

FIG. 3 is a photographic and graphical representation showing that CQ does not affect polyQ mRNA levels or protein degradation. Bars and error bars represent mean±s.e.m. n≧3 for each data set. (a) Northern blotting and quantitation of htt^{exon1}-Q¹⁰³-egfp transcripts. (b) Fluorescence microscopy of mutant protein accumulation in htt^{exon1}−Q¹⁰³-egfp-expressing cells in presence of MG132. Scale bar, 100 μm. (c) Western analysis of cells treated with MG132 and CQ. (d) Pulse-chase analysis of htt^{exon1}-Q¹⁰³-egfp turnover in the presence and absence of CQ.

FIG. 4 is a photographic and graphical representation showing that CQ inhibits mutant Htt aggregate accumulation in vivo. Bars and error bars represent mean+s.e.m. (a) Representative western blotting and quantitation of high molecular weight mutant Htt in whole brain homogenates from 11-week old R6/2 transgenic mice. Wt, wild-type; Tg, vehicle-treated R6/2; CQ, CQ-treated R6/2. n=3. *P=0.01. (b) Inhibition of neuronal intranuclear inclusion formation by treatment with CQ. Representative images of striatum and cortex from 1′-week old mice; wild-type, R6/2-vehicle treated, R6/2-CQ treated, as indicated. Htt aggregates were visualized using EM48 Ab. Results were similar for six or more animals in each treatment group. Scale bar, 50 μm.

FIG. 5 is a photographic and graphical representation showing that CQ decreases polyQ-mediated pathology in vivo. Bars and error bars represent mean+s.e.m. Representative images of the cerebrum of 1′-week old mice and quantitation of lateral ventricle areas, showing decreased striatal atrophy in 11-week old CQ-treated R6/2 mice. n≧6 for each treatment group. *P<0.001 versus vehicle-treated mice.

FIG. 6 is a photographic and graphical representation showing that CQ positively effects behavior, weight and survival in vivo. Bars and error bars represent mean+s.e.m. (a) Illustration of clasping behavior, left, wild-type mouse, right, 10 week-old R6/2 mouse exhibiting clasping. (b) Clasping score at 10 weeks of age in CQ and vehicle-treated R6/2. n=8. *P<0.0004 versus vehicle. (c) Rotarod testing of CQ and vehicle-treating R6/2. Bars and error bars represent mean+s.e.m. black bars, vehicle-treated, grey bars, CQ-treated. n≧6 for each group. *P<0.03, **P<0.001 versus vehicle-treated animals. (d) Body weight over time of wild type (filled circles), vehicles-treated R6/2 (open circles) and CQ-treated R6/2 (filled triangles); symbols and bars represent mean±s.e.m. n≧6 for each group, *P<0.01 versus vehicle-treated animals by Studient-t test; also, P<0.0001 overall, for CQ-treated versus vehicle-treated animals by ANOVA with post-hoc Bonferroni/Dunn testing. (e) Kaplan-Meier analysis of R6/2 lifespan, vehicle treated (closed circles) versus CQ treated (open circles). n=5 per group. P=0.0018 by log-rank, Mantel-Cox test.

### DETAILED DESCRIPTION OF THE INVENTION

Abbreviations and Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e. unbranched) or branched chain, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

Typically, an alkyl group will have from 1 to 24 carbon atoms, with those groups having 10 or fewer carbon atoms being preferred in the present invention. A “lower alkyl” is a shorter chain alkyl, generally having eight or fewer carbon atoms.

The terms “halo” or “halogen,” mean, unless otherwise stated, a fluorine, chlorine, bromine, or iodine atom.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, -SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R“ ” each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound of the invention includes more than one R group, for example, each of the R groups is independently selected as are each R′, R″, R′″ and R“ ” groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R“ ” are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the invention includes more than one R group, for example, each of the R groups is independently selected as are each R′, R″, R′″ and R“ ” groups when more than one of these groups is present.

Two of the substituents on adjacent atoms of the aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (eg (+)-tartrates, (−)-tartrates or mixtures thereof including racemic mixtures), succinates, benzoates and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, tautomers, geometric isomers and individual isomers are encompassed within the scope of the present invention. The compounds of the present invention do not include those which are known in the art to be too unstable to synthesize and/or isolate.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

It must be noted that, as used in the subject specification, the singular forms “a”, “an” and “the” include plural aspects unless the context clearly dictates otherwise. Thus, for example, reference to “an active agent” includes a single active agent, as well as two or more active agents; reference to “a disease condition” includes a single disease condition as well as two or more disease conditions; and so forth.

The term “polyglutamine-expression disease” or “polyQ-expansion disease” includes any condition involving an expanded CAA and/or CAG repeat region in a gene which results in the generation of polyQ. In the case of HD, the polyQ is part of the Htt protein. PolyQ-expansion disease can result in transcriptional dysregulation, activation of apoptotic pathways, energy failure, excitotoxicity (i.e. over activation of NMDA receptors) and loss of trophic support.

The term “neurological condition” is used herein in its broadest sense and refers to conditions in which various cell types of the nervous system are degenerated and/or have been damaged as a result of neurological disorders or injuries or exposures.

An “effective amount” of a compound of the present invention is the amount effective to achieve its intended purpose. The term “effective amount,” when used in the context of a method of treating a subject (e.g. a mammal, human, etc.) is a therapeutically effective amount (i.e. the amount effective to achieve therapeutically effective results against the stated disease or disease state). “Treatment” or “treating” includes prevention and amelioration of a particular disease or disease state or symptoms thereof, and in some cases, curing a disease. A “therapeutically effective amount” includes a “prophylactically effective amount.” The specific “therapeutically effective amount” will of course vary with such factors as the particular condition being treated, the physical condition and clinical history of the subject, the type of animal being treated, the duration of the treatment, the nature of concurrent therapy (if any), and the specific formulations employed and the structure of the compound or its derivatives.

Abbreviations used herein are defined in Table 1 below.

TABLE 1

| Abbreviation | Definition |
|---|---|
| BDNF | Brain-derived neurotrophic factor |
| CQ | Clioquinol (5-chloro-7-iodo-8-hydroxy-quinoline) |
| EGFP | Enhanced green fluorescent protein |
| egfp | Genetic sequence encoding EGFP |
| GFP | Green fluorescent protein |
| HD | Huntington's disease |
| Htt | Huntingtin protein |
| htt | Gene encoding Htt |
| MCI | Mild Cognitive Impairment |
| polyQ | Polyglutamine |
| ROS | Reactive oxygen spcies |
| SMON | Subacute myelo-optic neuropathy |
| wt | Wild-type |

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of the sequence identifiers is provided in Table 2. A paper copy of the Sequence Listing and a computer readable form of the sequence listing on compact disk is submitted herewith and are herein incorporated by reference.

Genetic sequences are represented in italics and in lower case whilst proteins are non-italicized and with the first letter in upper case. For example, the gene encoding huntingtin protein (Htt) is written as htt.

TABLE 2

| SEQUENCE | |
|---|---|
| ID NO: | DESCRIPTION |
| 1 | CAA- and CAG- containing tandem repeats of |
| | polyglutamine region in polyQ-htt-GFP fusion construct |
| 2 | FLAG introduced into GFP in Q15 HDexon I construct |

### I. Methods of Treating a Polyglutamine (polyQ) Expansion Diseases using an 8-Hydroxyquinoline Compound

The present invention provides compounds and methods for the treatment and/or prophylaxis of neurological disorders and/or conditions associated with polyQ-expansion disease. Such disorders or conditions are neurological disorders or conditions which include any neurological state which results in or otherwise contributes to motor, cognitive and psychiatric dysfunction. In particular, the present invention contemplates a method for the treatment or prophylaxis of a polyQ-expansion disease including but not limited to HD, spinal and bulbar muscular atrophy, dentatoribral-pallidoluysian atrophy, spinocerebellar ataxic type I and Machado-Joseph disease. Preferred agents of the present invention are 8-hydroxyquinoline compound metal binding agents which sequester or bind to metal ions. One example is iodochlorhydroxyquin (also known as clioquinol [CQ] or 5-chloro-7-iodo-8-hydroxy-quinoline or iodochlorhyrdoxyquin), which is a bio-available Cu/Zn binding agent. Reference herein to “CQ” or its other names includes all functional derivatives, analogs, homologs, isomeric and tautomeric forms thereof. It is proposed that CQ is useful in reducing oxidative stress and the production of ROS. Although not limiting the invention to any one theory, it is proposed that CQ interacts with RNA or protein to modulate translation of Htt.

In one aspect, the present invention provides methods of treating a polyglutamine (poly Q) expansion disease in a subject in need of such treatment. The method includes administering to the subject an effective amount of an 8-hydroxyquinoline compound.

In certain embodiments, the 8-hydroxyquinoline compound useful in the methods of the present invention is a Cu⁺⁺—Zn⁺⁺-binding 8-hydroxyquinoline compound. The compound is typically capable of penetrating cells and reducing the production of reactive oxygen species (ROS). In some embodiments, the 8-hydroxyquinoline compound is capable of crossing the blood-brain barrier, enhancing Aβ aggregate dissolution, decreasing Aβ toxicity, and/or safe for administration to the subject under conditions of controlled dosing and vitamin supplementation.

In some embodiments, the 8-hydroxyquinoline compound useful in the methods of the present invention has the formula:

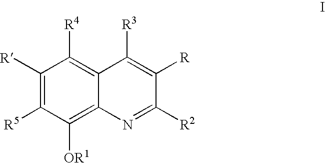

In Formula I, R¹ is H, —C(O)R A^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an antioxidant, or a targeting moiety. R^{1A} is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R² is H, substituted or unsubstituted alkyl, substituted or unsubstituted alkoxy, substituted or unsubstituted heteroalkyl, substituted or unsubstituted cycloalkyl, substituted or unsubstituted heterocycloalkyl, substituted or unsubstituted aryl, an antioxidant; a targeting moiety, —C(O)R⁶ or —C(S)R⁶. R⁶ is H, substituted or unsubstituted alkyl, hydroxy, substituted or unsubstituted aryl, substituted or unsubstituted heteroaryl, substituted or unsubstituted heterocycloalkyl, an antioxidant, a targeting moiety, —OR⁷, —SR⁷, or —NR⁷R⁸. R⁷ and R⁸ are independently H, substituted or unsubstituted alkyl, substituted or unsubstituted, substituted or unsubstituted heteroaryl, substituted or unsubstituted heterocycloalkyl, CN, CH₂NR⁹R¹⁰, HCNOR⁹, or HCNNR⁹R¹⁰. R⁹ and R¹⁰ are independently H, substituted or unsubstituted alkyl, substituted or unsubstituted aryl, substituted or unsubstituted heteroaryl, substituted or unsubstituted heterocycloalkyl, OR¹¹, SR¹¹ or NR¹¹R¹². R¹¹ and R¹² are independently H, substituted or unsubstituted alkyl, substituted or unsubstituted aryl, substituted or unsubstituted heteroaryl, substituted or unsubstituted heterocycloalkyl, together form optionally substituted heterocyclyl, or SO₂NR¹³R¹⁴. R¹³ and R¹⁴ are independently H, substituted or unsubstituted alkyl, substituted or unsubstituted heteroaryl, or substituted or unsubstituted heterocycloalkyl.

R³, R⁴, R⁵, R and R′ are independently H, substituted or unsubstituted alkyl, substituted or unsubstituted alkoxy, —C(O)R¹⁵, hydroxy, alkylamino, alkylthio, alkylsulphonyl, alkylsulphinyl, halo, —SO₃H, amine, substituted or unsubstituted aryl, substituted or unsubstituted heteroaryl, or substituted or unsubstituted heterocycloalkyl, an anti-oxidant, or a targeting moiety. R¹⁵ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In a preferred embodiment, R, R², and R³ are H. In another preferred embodiment, R, R¹, R², and R³ are H. In some related embodiments, R⁴ and R⁵ are independently halogen, or unsubstituted C₁-C₁₀ alkyl. In another related embodiment, R⁴ and R⁵ are independently halogen, or unsubstituted C₁-C₆ alkyl. In another related embodiment, R⁴ and R⁵ are independently halogen, or unsubstituted C₁-C₃ alkyl. In another related embodiment, R⁴ and R⁵ are independently halogen or methyl. In another related embodiment, R⁴ and R⁵ are independently Cl or I. In another related embodiment, R⁴ is Cl and R⁵ is I.

In another preferred embodiment, R¹ is H or unsubstituted C₁-C₁₀ alkyl. R¹ may also be H or unsubstituted C₁-C₆ alkyl. In certain embodiments, R′ is H or unsubstituted C₁-C₃ alkyl. In other embodiments, R¹ is H or methyl.

In another preferred embodiment, R, R¹, R², and R³ are H; R¹ is H or unsubstituted C₁-C₁₀ alkyl; and R⁴ and R⁵ are independently halogen or unsubstituted C₁-C₁₀ alkyl. In another preferred embodiment, R, R¹, R², and R³ are H; R¹ is H or unsubstituted C₁-C₆ alkyl; and R⁴ and R⁵ are halogen or unsubstituted C₁-C₆ alkyl. In another preferred embodiment, R, R¹, R², and R³ are H; R′ is H or unsubstituted C₁-C₃ alkyl; and R⁴ and R⁵ are independently halogen or unsubstituted C₁-C₃ alkyl. In another preferred embodiment, R, R¹, R², and R³ are H; R¹ is H or methyl; and R⁴ and R⁵ are independently halogen or methyl. In another preferred embodiment, R, R¹, R², and R³ are H; R¹ is H; and R⁴ and R⁵ are independently halogen. In another preferred embodiment, R, R¹, R², and R³ are H; R′ is H; and R⁴ and R⁵ are independently Cl or 1.

In another preferred embodiment, the 8-hydroxyquinoline compound is CQ.

In some embodiments, the present invention provides a method for the treatment or prophylaxis of HD or another polyQ-expansion disease in a subject, said method comprising administering to said subject an effective amount of 8-hydroxyquinoline compound (e.g. CQ or a derivative, analog, homolog, isomeric or tautomeric thereof) for a time and under conditions sufficient to ameliorate the symptoms of HD or other polyQ-expansion disease.

Another embodiment of the present invention provides a method for ameliorating the symptoms of HD or other polyQ-expansion disease in a subject. The method comprises administering to said subject an effective amount of 8-hydroxyquinoline compound (e.g. CQ or a derivative, analog, homolog, isomeric or tautomeric thereof) for a time and under conditions sufficient to ameliorate symptoms of cognitive, motor and/or psychiatric dysfunction associated with said polyQ-expansion disease.

Still another embodiment of the present invention is directed to the use of 8-hydroxyquinoline compound (e.g. CQ or a derivative, analog, homolog, isomeric or tautomeric thereof) in the manufacture of a medicament for the treatment of HD or other polyQ-expansion disease or to ameliorate symptoms thereof including motor, cognitive and/or psychiatric dysfunction in a subject.

Still another embodiment of the present invention provides a therapeutic composition or agent for use in treating HD or other polyQ-expansion disease in a subject. The composition comprises an 8-hydroxyquinoline compound (e.g. CQ or a derivative, analog, homolog, isomeric or tautomeric thereof) and one or more pharmaceutically acceptable carriers, diluents, excipients and/or other active agents.

Preferred subjects include mammals such as humans.

The present invention also include methods directed to the use of an 8-hydroxyquinoline compound (e.g. CQ or a derivative, analog, homolog, isomeric or tautomeric thereof) as a target for the design of agonists and mimetics and/or related compounds or derivatives all useful in the treatment of polyQ-expansion disease.

Examples of polyQ-expansion diseases include HD, spinal and bulbar muscular atrophy (Kennedy's disease), dentatorubral-pallidoluysian atrophy, spinocerebellar ataxia (SCA) types 1, 2, 3(Machado-Joseph disease), 7 and 12.

It is to be understood that unless otherwise indicated, the subject invention is not limited to specific formulations of components, manufacturing methods, dosage regimens, or the like, as such may vary. It is also to be understood that the terminology used herein is for the purpose of describing particular embodiments only and is not intended to be limiting.

In some embodiments, the 8-hydroxyquinoline compound is useful in improving cognitive function and reducing oxidative stress. The expression “improving cognitive function” as used herein means slowing or arresting decline in cognitive function, increasing cognitive functioning, preventing or deferring the onset of cognitive dysfunction, relative to age-matched controls. Cognitive function may suitably be assessed by tests well known in the art, such as the ADAS-cog test, or other conventional cognitive screening tests, such as the Mini Mental Status Exam, and the Memory Impairment Screen. A more powerfully discriminating cognitive test, such as the CogState test (CogState Ltd, www.cogstate.com), may also be used. The expression “oxidative stress” refers to the process whereby the amount of free radicals or reactive oxygen species increase, subsequent cell damage occurs and disease results. Free radicals are aggressive atoms or molecules that cause damage when they react with cell components. They are highly reactive due to unimpaired electrons. Free radicals attack the nearest stable molecule and sequester its electron, thereby oxidizing the molecule. Indicators of oxidative stress caused by free radicals include damaged DNA bases, protein oxidation products and lipid peroxidation products.

The term “metal binding agent” is used herein in its broadest sense, and refers to compounds having two or more donor atoms capable of binding to a metal atom, preferably copper, zinc or iron. In one particular embodiment, such as in the treatment of HD, the metal binding agent will have a higher thermodynamic stability than that of huntingtin (Htt). Preferred forms of copper, zinc and iron are Cu, Zn and Fe ions such as Cu²⁺, Zn²⁺ and Fe³⁺. The metals may be referred to herein by their full name or two letter abbreviation.

The term “a specific metal binding agent” as used herein refers to CQ or a functional derivative, analog, homolog, isomeric or tautomeric form thereof, which has the ability to bind to, for example, Zn²⁺, Cu²⁺ or Fe³⁺ ions.

The terms “compound”, “agent”, “pharmacologically active agent”, “medicament”, “active” and “drug” are used interchangeably herein to refer to a chemical compound that induces a desired pharmacological and/or physiological effect. The terms also encompass pharmaceutically acceptable and pharmacologically active ingredients of those active agents specifically mentioned herein including but not limited to salts, esters, amides, prodrugs, active metabolites, analogs and the like. When the terms “compound”, “active agent”, “pharmacologically active agent”, “medicament”, “active” and “drug” are used, then it is to be understood that this includes the active agentper se as well as pharmaceutically acceptable, pharmacologically active salts, esters, amides, prodrugs, metabolites, analogs, etc.

Reference to a “compound”, “agent”, “pharmacologically active agent”, “medicament”, “active” and “drug” includes combinations of two or more actives such as one or more metal ion metal binding agents. A “combination” also includes a two or more-part including a multi-part pharmaceutical composition where the agents are provided separately and given or dispensed separately or admixed together prior to dispensation. In one example, the 8-hydroxyquinoline compound (e.g. CQ) is given in combination with agents which reduce cholesterol levels or which ameliorate other symptoms of neurological dysfunction. In addition, the 8-hydroxyquinoline compound (e.g. CQ) may be given simultaneously or sequentially (in either order) with histone deacetylase inhibitors such as suberoylanalide hydroxamic acid (SAHA) and sodium butyrate, caspase inhibitors such as 2VAD-fmk and minocycline, mitochondrial/energy modulators such as creatine and coenzyme Q₁₀, antiexcitotoxcity agents such as remacemide and riluzole, anti-aggregate agents such as congo red and trehalose. Compositions comprising the 8-hydroxyquinoline compound (e.g. CQ) and one or more of the aforementioned compounds are also contemplated as part of the present invention.

Accordingly, another aspect of the present invention is directed to a composition comprising an 8-hydroxyquinoline compound (e.g. CQ) and at least one other agent selected from suberoylanalide hydroxamic acid (SAHA), sodium butyrate, 2VAD-fmk, minocycline, creatine, coenzyme Q₁₀, riluzole, congo red and trehalose said composition further comprising one or more pharmaceutically acceptable carriers and/or diluents.

The compositions and agents described herein are given in a therapeutically effective amount to treat a polyQ-expansion disease or to ameliorate its symptoms.

In addition, the 8-hydroxyquinoline compound (e.g. CQ) may be given in conjunction with RNAi to reduce expression of polyQ-htt. Furthermore, molecules immunointeractive to polyQ such as monoclonal or polyclonal antibodies or catalytic antibodies may be employed. Still furthermore, screening of natural products or chemical libraries is proposed to identify small molecule inhibitors of polyQ-Htt translation. Similar screening is proposed to identify agonists, mimetics and chemical or functional equivalents of the 8-hydroxyquinoline compound (e.g. CQ).

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emusifying agents, pH buffering agents, preservatives, and the like.

Similarly, a “pharmacologically acceptable” salt, ester, emide, prodrug or derivative of a compound as provided herein is a salt, ester, amide, prodrug or derivative that this not biologically or otherwise undesirable. The carrier may be liquid or solid, and is selected with the planned manner of administration in mind.

The 8-hydroxyquinoline compound of the invention may be administered orally, topically, or parenterally in dosage unit formulations containing conventional non-toxic pharmaceutically acceptable carriers, adjuvants, and vehicles. The term parenteral as used herein includes subcutaneous, intravenous, intramuscular, intrathecal, intracranial, injection or infusion techniques.

Generally, the terms “treating”, “treatment” and the like are used herein to mean affecting a subject, tissue or cell to obtain a desired pharmacological and/or physiological effect. The effect may be prophylactic in terms of completely or partially preventing a disease or sign or symptom thereof, and/or may be therapeutic in terms of a partial or complete cure of a disease. “Treating” as used herein covers any treatment of, or prevention of disease, and includes preventing the disease from occurring in a subject which may be predisposed to the disease, but has not yet been diagnosed as having it; inhibiting the disease; i.e. arresting its development; or relieving or ameliorating the effects of the disease; i.e. causing regression of the effects of the disease.

The term “subject” as used herein refers to any animal having a disease or condition which requires treatment with a pharmaceutically-active agent. The subject may be a mammal, preferably a primate and most preferably a human, or may be a domestic or companion animal. While it is particularly contemplated that the compound of the present invention is suitable for use in medical treatment of humans, it is also applicable to veterinary treatment, including treatment of companion animals such as dogs and cats, and domestic animals such as horses, ponies, donkeys, mules, llama, alpaca, pigs, cattle and sheep, or zoo animals such as primates, felids, canids, bovids and ungulates.

Suitable mammals include members of the Orders Primates, Rodentia, Lagomorpha, Cetacea, Camivora, Perissodactyla and Artiodactyla. Members of the Orders Perissodactyla and Artiodactyla are particularly preferred because of their similar biology and economic importance.

For example, Artiodactyla comprises approximately 150 living species distributed through nine families: pigs (Suidae), peccaries (Tayassuidae), hippopotamuses (Hippopotamidae), camels (Camelidae), chevrotains (Tragulidae), giraffes and okapi (Giraffidae), deer (Cervidae), pronghom (Antilocapridae), and cattle, sheep, goats and antelope (Bovidae). Many of these animals are used as feed animals in various countries. More importantly, many of the economically important animals such as goats, sheep, cattle and pigs have very similar biology and share high degrees of genomic homology.

The Order Perissodactyla comprises horses and donkeys, which are both economically important and closely related. Indeed, it is well known that horses and donkeys interbreed.

Subjects requiring treatment for psychiatric dysfunction frequently display a level of cognitive impairment such as pre- or mild cognitive impairment or memory loss, varying from moderate to severe. In some embodiments, the 8-hydroxyquinoline compounds of the present invention ameliorate the symptoms of cognitive impairment such as pre- or mild cognitive impairment or memory loss as well as motor impairment and psychiatric dysfunction. The 8-hydroxyquinoline compound may, therefore, be given in conjunction with other therapeutic protocols for treating neurological or psychiatric conditions.

In a particularly preferred embodiment, the levels of free radicals or reactive oxygen species in subjects exhibiting a moderate to severe polyQ-expansion disease are reduced in the presence of the 8-hydroxyquinoline compound.

It may be advantageous for the 8-hydroxyquinoline compound to be able to be concentrated within the central nervous system. This capability may be designed into the 8-hydroxyquinoline compound through the inclusion of groups that enable the agent to be actively or passively transported into the brain, for example by formation of a lipophilic diester (See Australian Patent No. 739835).

It may also be advantageous for a single molecule of the 8-hydroxyquinoline compound to be able to provide three or more chelation points to enable a 1:1 ratio of agent:metal ion.

The present invention also provides for the use of the 8-hydroxyquinoline compounds and compositions disclosed herein as neurotherapeutic or neuroprotective agents, comprising an 8-hydroxyquinoline compound for the treatment and/or prophylaxis of a polyQ-expansion disease such as HD.

Further, the present invention contemplates the use of the 8-hydroxyquinoline compounds and compositions disclosed herein in the manufacture of a medicament for use in the treatment and/or prophylaxis of a polyQ-expansion disease such as HD.

In all aspects of the present invention the 8-hydroxyquinoline compound is preferably administered in a dosage range of 100 to 1,500 mg/day, more preferably 250 to 750 mg/day such as 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740 or 750 mg/day. This may optionally be administered in a divided dose. Furthermore, the dosage may be adjusted so that it is given per two days, per three days, per four days, per five days or per week or per month. In addition, the 8-hydroxyquinoline compound may be given sequentially or simultaneously with other active agents. The amounts of the other active agents are similar or comparable to the above amounts.

In addition to slowing or arresting the symptoms of a polyQ-expansion disease, the methods and 8-hydroxyquinoline compounds of the present invention may also be suitable for use in the treatment or prevention of associated conditions or secondary symptoms such as cognitive, motor or psychiatric decline including dementias.

Dementias are usually not diagnosed until one or more warning symptoms have appeared. These symptoms constitute the MCI syndrome as defined by the American Academy of Neurology, and refers to the clinical state of individuals who have memory impairment, but who are otherwise functioning well, and who do not meet clinical criteria for dementia (Petersen et al., Neurology 56: 1133-1142, 2001). It is generally accepted that MCI is a precursor of many diseases associated with increased levels of free radicals such as AD and Parkinson's disease and may also be a precursor of dementias resulting from other pathological causes. Symptoms of MCI include: memory loss which affects job skills; difficulty performing familiar tasks; problems with language; disorientation as to time and place (getting lost); poor or decreased judgement; problems with abstract thinking; misplacing things; changes in mood or behaviour; changes in personality; and/or loss of initiative.

MCI can be detected using conventional cognitive screening tests, such as the Mini Mental Status Exam, and the Memory Impairment Screen and neuropsychological screening batteries.

The 8-hydroxyquinoline compound and compositions of the present invention may be administered by any suitable route and the person skilled in the art will readily be able to determine the most suitable route and dose for the condition to be treated. Dosage will be at the discretion of the attendant physician, and will depend on the nature and state of the condition to be treated, the age and general state of health of the subject to be treated, the route of administration, and any previous treatment which may have been administered.

Methods and pharmaceutical carriers for preparation of pharmaceutical compositions are well known in the art, as set out in textbooks such as Remington's Pharmaceutical Sciences, 20^{th} Edition, Williams & Wilkins, Pennsylvania, USA. The carrier or diluent and other excipients will depend on the route of administration and, again, the person skilled in the art will readily be able to determine the most suitable formulation for each particular case.

The 8-hydroxyquinoline compounds of the present invention may optionally be administered in conjunction with one or more other pharmaceutically active agents suitable for the treatment of the condition; i.e. it may be given together with, before, or after one or more such agents. For example, where the condition is a HD condition, the compound may be used in conjunction with treatment with another agent, such as histone deacetylase inhibitors such as suberoylanalide hydroxamic acid (SAHA) and sodium butyrate, caspase inhibitors such as 2VAD-fmk and minocycline, mitochondrial/energy modulators such as creatine and coenzyme Q₁₀, antiexcitotoxicity agents such as remacemide and riluzole, anti-aggregate agents such as congo red and trehalose, an inhibitor of the acetylcholinesterase active site, for example, phenserine, galantamine, or tacrine; an anti-oxidant, such as Vitamin E or Vitamin C; an anti-inflammatory agent, such as flurbiprofen or ibuprofen, optionally modified to release nitric oxide (for example, NCX-2216, produced by NicOx), or an oestrogenic agent such as 17-β-oestradiol.

The present invention includes the use of various pharmaceutical compositions useful for ameliorating disease. The pharmaceutical compositions may be prepared by bringing the 8-hydroxyquinoline compound and optionally one or more other pharmaceutically active agents, and/or other active agents agent into a form suitable for administration to a subject, using carriers, excipients and additives or auxiliaries.

Frequently-used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include anti-microbials, anti-oxidants, metal binding agents and inert gases. Other pharmaceutically acceptable carriers include aqueous solutions, non-toxic excipients, including salts, preservatives, buffers and the like, as described, for instance, in Remington's Pharmaceutical Sciences, 2000, Supra and The British National Formulary 43^{rd} Ed. (British Medical Association and Royal Pharmaceutical Society of Great Britain, 2002; <http://bnf.rhn.net>). The pH and exact concentration of the various components of the pharmaceutical composition are adjusted according to routine skills in the art; see Goodman and Gilman's “The Pharmacological Basis for Therapeutics” (7^{th} Ed., 1985).

The pharmaceutical compositions are preferably prepared and administered in dosage units. Solid dosage units include tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

### II. Assays

Candidate 8-hydroxyquinoline compounds useful in methods of treating a (poly Q) expansion disease may be identified in vitro and subsequently tested in cellular and/or animal (e.g. mouse) models for various effects related to poly Q expansion diseases. The activity of the inhibitor compounds can be assayed utilizing methods known in the art and/or those methods presented herein, including the Examples section below.

For instance, a candidate 8-hydroxyquinoline compound can be tested for its ability to selectively down-regulate expanded polyglutamine in cells transfected with the Htt^{exon1}-Q¹⁰³-egfp plasmid. Using fluorescence microscopy, EGFP, htt^{exon1}-Q²⁵-egfp and htt^{exon1}-Q¹⁰³-egfp iexpressing cells are quantitated. The ability of a candidate 8-hydroxyquinoline compound to selectively down-regulate polyglutamine protein expression may be tested by transiently transfecting cells expressing Htt exon-1 containing a 103 residue polyglutamine-encoding region fused to an enhanced green fluorescent protein gene.

The ability of a candidate 8-hydroxyquinoline compound to inhibit mutant Htt aggregate accumulation in vivo may be assayed using an HD transgenic mouse model and measuring the reduction in the quantity of aggregated huntingtin, as indicated by western blotting of whole-brain extracts. Candidate 8-hydroxyquinoline compounds may also be assayed for their ability to decreases polyQ-mediated pathology in vivo using anti-Htt monoclonal antibody to detect striatal atrophy in cryosectioned brain in a mouse model. The effect of a candidate 8-hydroxyquinoline compound may on animal model behavior, weight and survival of a may also be assayed using the Rotarod performance test as described by Hockly et al., 2003.

As set forth in Example 17 below, high throughput screening of candidate 8-hydroxyquinoline compounds may be performed using a dual reporter protein containing a C-terminal green and N-terminal red fluorescent protein and an intervening polyQ segment to test the ability of a compound to selectively inhibit transcription or translation of expanded CAG repeats.

Thus, the present invention further provides an animal model for studying HD or other polyQ-expansion diseases. Such animal models are useful for screening for agonists or mimetics of CQ or CQ itself or polyQ-Htt may be used as a target for small molecule screening or the development of antibodies specific for the polyQ domain of mutated Htt.

### III. EXAMPLES

The following examples are for illustrative purposes only and are not intended to limit the scope of the invention.

In investigating the role of metal ions in the pathogenesis of Huntington's disease (HD), the effects of clioquinol (CQ) were examined on mutant huntingtin (htt)-expressing cells. PC12 cells expressing polyglutamine (polyQ)-expanded htt exon 1 accumulated less mutant protein and showed decreased cell death when treated with CQ. This effect was polyQ-length specific, and did not alter mRNA levels or protein degradation rates, suggesting that decreased expression might be due to inhibition of translation. CQ treatment of transgenic Huntington's mice (R6/2) improved behavioral and pathologic phenotypes including decreased htt aggregate accumulation, decreased striatal atrophy, improved rotarod performance, reduction in weight loss, normalisation of blood glucose and insulin levels, and extension of lifespan.

### Example 1

R6/2 Transgenic and Wild-Type Littermate Mice

A colony of transgenic mice strain R6/2 that is heterozygous for htt exon1 (containing 145 CAG repeats) was maintained, with founders originating and available from The Jackson Laboratory (Bar Harbor, Me., USA). Male transgenic R6/2 mice were bred with background strain (B6CBAFI/J) females. Genotyping was done according to Hockly et al, Brain Res Bull 61, 469-479, 2003. CQ in water was orally administered to mice at 30 mg/kg/day starting at 3 weeks of age and continued until the mice were deceased or sacrificed for tissues. Littermate R6/2 mice were gavaged with water vehicle alone. At the end of CQ treatment mice were anesthetized and transcardially perfused with PBS followed by 4% v/v paraformaldehyde. Blood glucose levels were determined after at least 8 hrs of fasting in 11-week old mice (n=6 for wild-type, n=4 for vehicle-treated R6/2, n=6 for CQ-treated R6/2 littermates) using a 2300 STAT Plus glucose analyzer (YSI, Yellowsonte, Ohio, USA). Fasting blood insulin levels in animals fasted for 8 hrs were measured using an Ultra Sensitive Rat Insulin ELISA Kit (Crystal Chem, Inc., Downers Grove, Ill., USA) according to manufacturer's instructions.

### Example 2

Cell Culture, Transfection and CQ Treatment

PolyQ-htt-GFP fusion constructs, in which the polyglutamine region is encoded by CAA and CAG-containing tandem repeats (CAACAGCAACAACAGCAG) [SEQ ID NO: 1], human polyglutaminen, were obtained from the Hereditary Disease Foundation, Santa Monica, Calif., USA. PC12 and HEK293 cells were maintained in MEM supplemented with 10% v/v FBS, switched to Opti-MEM without serum for 6 hrs during transfection using Lipofectamine 2000 (Invitrogen, Carlsbad, Calif., USA), and returned to serum-containing medium thereafter (up to 48 hours). Cells were exposed to CQ (Sigma, St. Louis, Mo., USA) at 0.5 μM to 8 μM beginning 30 min prior to transfection. Control cultures received equivalent amounts of DMSO-containing vehicle (<0.1% w/v DMSO, final concentration).

### Example 3

Western Blotting

HEK293 or PC12 cells were lysed in PBS buffer containing 1% v/v Triton X-100 and protease inhibitor cocktail (Roche, Indianapolis, Ind.), and passed through a 28 gauge needle to ensure the release of Htt inclusions from the nuclei. Whole mouse brains were homogenized in 50 mM Tris-HCl (pH 7.4), 150 mM NaCl, 1% v/v Triton X-100 and complete protease inhibitor cocktail. Brain homogenate was pre-cleared by centrifuging at 7,500 g for 90 s at 4° C. The supernatant was passed through a 28-gauge needle and boiled in SDS sample buffer for 10 min. Fifty μg of total protein was loaded onto 10% w/v SDS gel containing a 4% w/v stacking gel. Monoclonal antibodies anti-polyglutamine (1:2,000), anti-GAPDH (1:10,000), anti-GFP (1:4,000), and anti-Htt (EM48) in 1:1000 dilution were used for immunoblotting. All western blot experiments were performed at least three times and band intensities were averaged.

### Example 4

Northern Analysis

Trizol reagent (Invitrogen, Carlsbad, Calif.) was used to isolate total RNA. Ten μg of total RNA was electrophoresed through 1.25% w/v agarose gel and transferred onto Hybond N+membrane. The membrane was baked in an 80° C. oven for 15 min and UV-crosslinked. Exon 1 of htt containing 103 CAA/CAG-repeats was used as a probe for northern blot detection according to AlkPhos Direct Labeling and Detection System (Amersham Biosciences, Piscataway, N.J.). One hundred ng of the probe was hybridized to the membrane in a 60° C. rotating hybridization oven overnight. The membrane was scanned using the Typhoon 9410 digital scanner (Amersham Biosciences, Piscataway, N.J.) to yield the signal image. The experiment was performed three times and band intensities were averaged.

### Example 5

Cell Viability and Section Measurement

Plasma membrane integrity was evaluated using propidium iodide at 20 μg/ml final concentration. Approximately 200 cells transfected with the Htt^{exon1}-Q₁₀₃-egf plasmid were counted for each treatment group 48 hr after transfection. Cells were at that time as expanded protein accumulation had reached a plateau, and 90% of cell death occurred by this time point. The experiment was repeated twice and counts were averaged. Counts of transfected cells were averaged from three independent, randomly-selected fields. ImageJ 1.32j (National Institutes of Health, USA), was used to measure the size of the lateral ventricles (n≧6 for each group).

### Example 6

Pulse-Chase Assay

HEK293 cells at 90% confluence on 100 mm plates coated with poly-D-lysine (0.1 μg/μl) were transfected for 4 hr in Opti-MEM medium before replacement of the medium with MEM supplemented with 10% v/v FBS. After another 5 hr incubation, cells were trypsinized and replated in two wells of a 6-well plate, then incubated for another 14 hr at 37° C. The medium was replaced with methionine-free MEM medium supplemented with 5% v/v FBS, and cells incubated for 1.5 hr with 4 μM of CQ or DMSO vehicle added during the last 30 min of incubation. One mCi of Smethionine (Amersham Biosciences, Piscataway, N.J.) in 12 ml of methionine-free MEM was prepared as a pulse stock medium. Cells were pulsed for 10 min by replacing the medium with 1 ml of the pulse stock medium. At different time points, cells were washed twice with ice-cold PBS and lysed in 1 ml of IP lysis buffer (50 mM Tris-HCl, pH 7.5, 150 mM NaCl, 1% v/v NP-40, 0.5% v/v sodium deoxycholate, 1 tab mini-complete protease inhibitor/10 ml buffer). Cells were harvested by scraping, placed in 1.5 ml microcentrifuge tube, and lysed with constant agitation at 4° C. for at least 30 min before immunoprecipitation of the Htt^{exon1}-Q₁₀₃-egfp protein using anti-polyglutamine antibody and protein G agarose beads (Roche, Indianapolis, Ind., USA). Assays were performed at least three times.

### Example 7

Brain Cryosectioning and Immunohistochemistry

Perfused mouse brains were fixed in 4% v/v paraformaldehyde for 1 hr at room temperature and stored in 30% w/v sucrose overnight at 4° C. Brain tissue was frozen at −70° C. for at least 30 min before cryosectioning at 40 μm thickness using a Leica CM1850 cryostat (McBain Instruments, Chatsworth, Calif.). Eleven-week old mouse brains (n>5 for each treatment group) sections were incubated at room temperature for 1 hr in blocking buffer containing 2% v/v goat serum, 0.2% v/v Triton X-100, and 0.1% v/v BSA in PBS. Then, EM48 anti-Htt monoclonal antibody (Chemicon, Temecula, Calif.) at a 1:100 dilution was incubated with the tissue at 4° C. overnight. Colorimetric signal detection was performed using DAB substrate and ABC kit (Vector Laboratories, Burlingame, Calif.) according to manufacturer's protocol.

### Example 8

Behavioral Tests

Rotarod performance (n≧6 for each treatment group) was assessed as described by Hockly et al., 2003 Supra with the modification that the SDI Rota-Rod (San Diego Instruments, San Diego, Calif.) was set to linearly increase in speed to 40 rpm in 4 min ramp time. For testing clasping, 10-week old mice (n=8 for each treatment group) were suspended by tail for 30 s and the foot-clasping time was scored such that a 0-5 s clasping duration was given a score of 1, 5-10 s a score of 2, and greater than 10 s a score of 3.

### Example 9

CQ Selectively Down-Regulates Polyglutamine Protein Expression and Relieves Cell Death in vitro

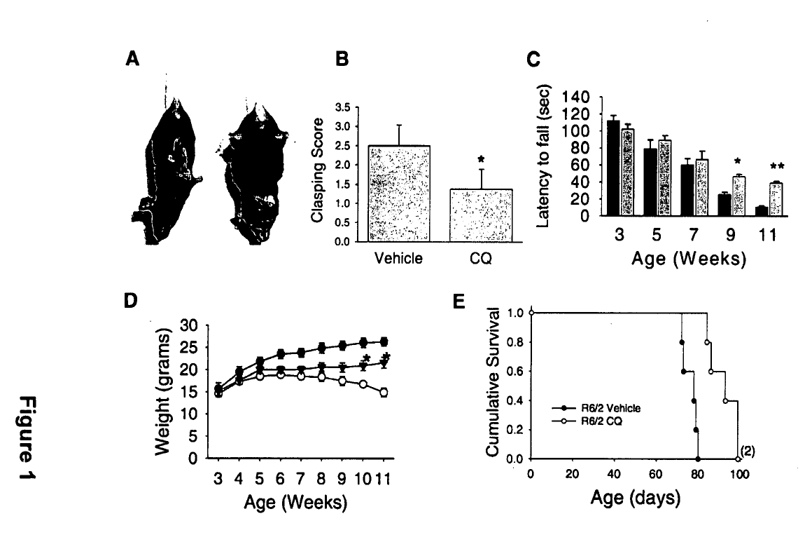

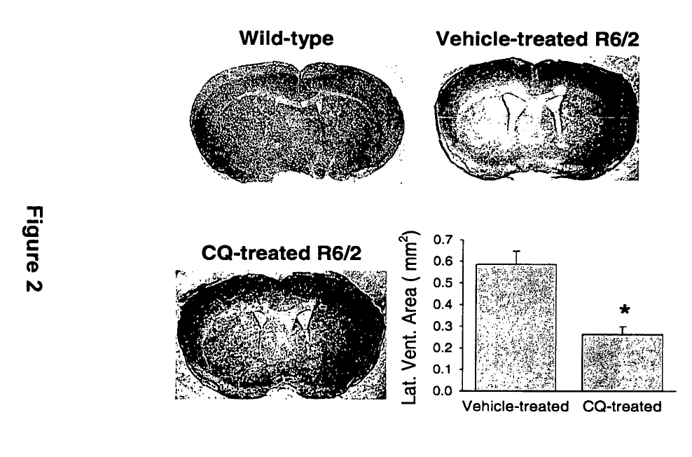

Transiently transfected PC12 cells expressing Htt exon-1 containing a 103 residue polyglutamine-encoding region fused to enhanced green fluorescent protein gene exon1_{-Q}_{₁₀₃}-egfp), produced less fluorescent product when treated with CQ at 4 μM, while Htt^{exon1}-Q25-egfp and egfp-alone were-unaffected at 48 hours post-transfection (FIG. 1). Western blotting of cell extracts showed a dose-dependent inhibition of Htt^{exon1}-Q₁₀₃-egfp expression beginning at 0.5 μM CQ and reaching 3.2 fold at 2 μM, which was not observed with egfp-alone (FIG. 2a). The lack of effects on Htt^{exon1}-Q₂₅-egfp and egfp alone suggested that CQ was not causing a generalized decrease in protein synthesis, a reduction in transfection efficiency, or affecting GFP maturation. Similar effects were observed at 6 and 12 hours post-transfection (not shown). Concordant with this reduction in mutant protein expression, cell survival was improved by CQ in a dose dependent fashion with a half maximal effect at 1-2 μM (FIG. 2b). Cell death began to increase at 8 μM, likely due to dose-dependent toxic effects of CQ.

### Example 10

CQ does not Affect PolyQ Protein Transcription or Degradation Rate

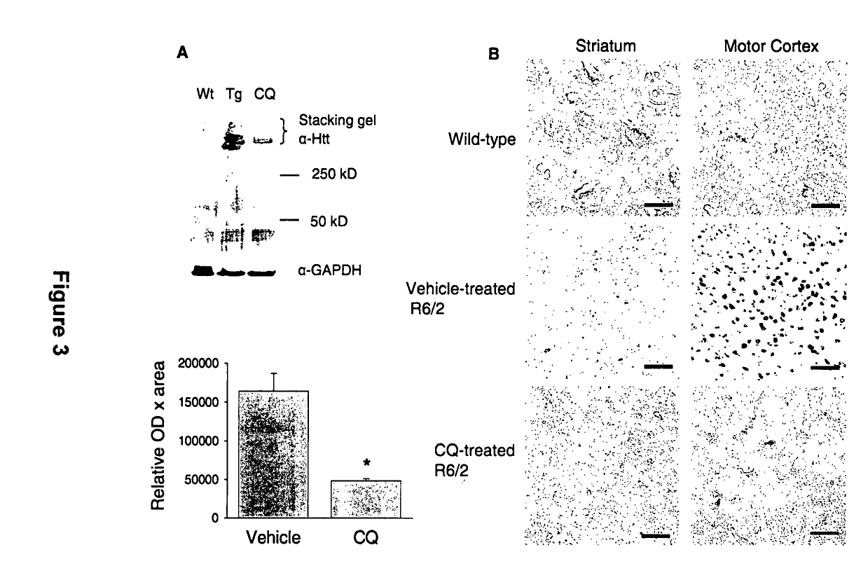

In examining potential mechanisms for the effects of CQ on Htt accumulation, we considered the possibilities that CQ might act to inhibit Htt aggregation or directly influence Htt synthesis or degradation. Since the early accumulation of Htt was inhibited by CQ, and did not result in a change in distribution of the accumulated material (FIG. 1), it appeared that the compound was not principally affecting aggregation. Therefore, we focused on the aspects of Htt synthesis and turnover. To determine whether CQ might down-regulate polyQ-Htt expression at the transcriptional level, we examined the amount of Htt^{exon1}-Q₁₀₃-egfp mRNA present after 48 hrs of transfection by Northern analysis. CQ had no significant effect on mRNA levels at concentrations that markedly diminish protein levels (FIGS. 3a and 2a). Since Htt-exon 1 is subject to ubiquitination and proteasomal degradation (Davies et al, Cell 90:537-548, 1997; Holmberg et al, Embo J 23:4307-4318, 2004), we examined the possibility that CQ might work by HttHtt enhancing proteasome-mediated turnover, by treating CQ-treated Htt^{exon1}-Q₁₀₃-egf-expressing HEK293 cells with the pan-proteasome inhibitor MG132. Suppression of ^{exon1}-Q₁₀₃-egf protein production was not affected by MG132 treatment (FIG. 3b,c). To further support this result and rule out enhanced degradation through alternative paths, pulse-chase experiments were performed. No significant differences in the rates of Q103-egfp degradation were observed in the presence of CQ (FIG. 3d), though there may have been a small trend towards decreased degradation at longer time points in CQ-treated cells.

### Example 11

### CQ Inhibits Accumulation of Aggregated Htt in vivo

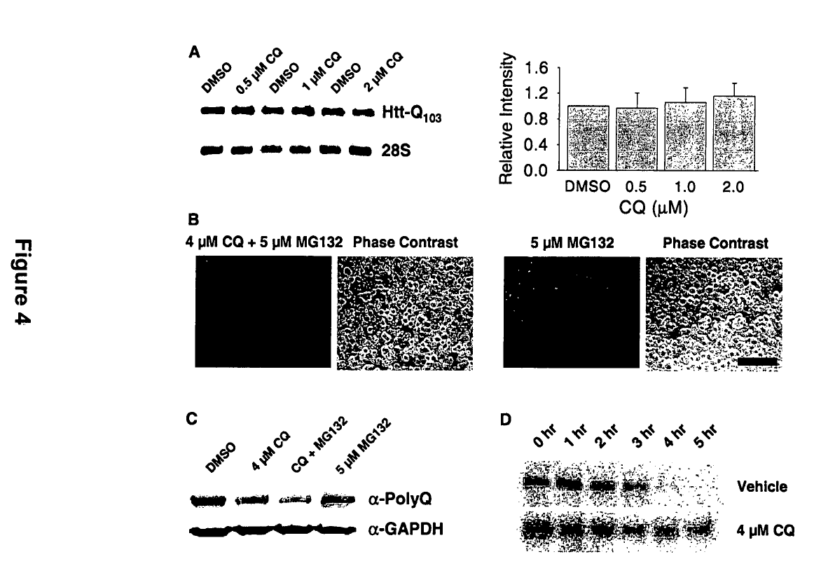

It was then determined if CQ would also reduce mutant Htt accumulation in vivo using the HD transgenic mouse model R6/2. After 8 weeks of daily oral CQ treatment, R6/2 mice had a 3.9-fold reduction in the quantity of aggregated huntingtin, as indicated by western blotting of whole-brain extracts (FIG. 4a). To examine the anatomical distribution of the changes in expression, immunohistochemistry with the anti-Htt aggregate Ab EM48 was performed, revealing that CQ treatment inhibited the accumulation of aggregated Htt in both the striatum and motor cortex (FIG. 4b).

### Example 12

CQ Treatment Improves HD Pathology and Enhances Motor Function and Survival in R6/2 Mice

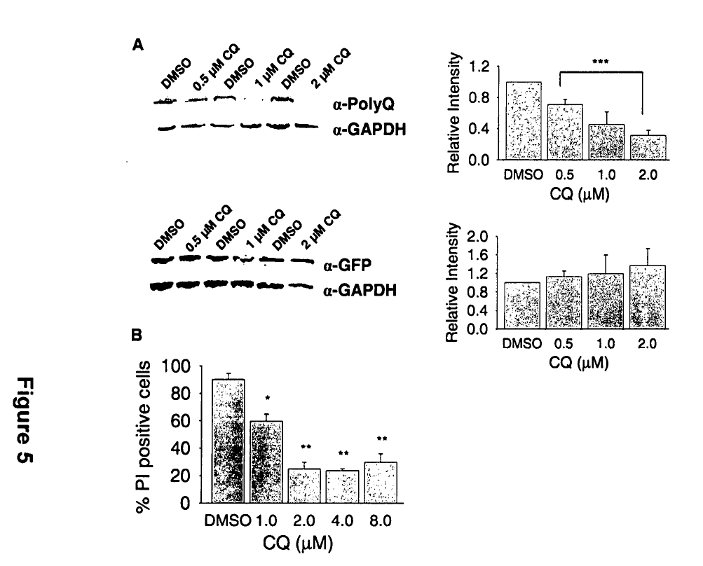

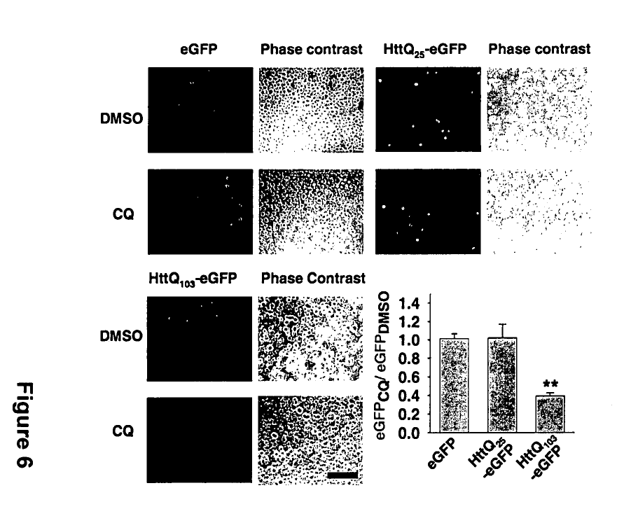

The major sites of cell loss in HD brain are the striatum and the cortex (Storey et al, Ann Neurol 32:526-534, 1992; Meade et al, J Comp Neurol 449:241-269, 2002) and in R6/2 mice, striatal atrophy is reflected by expansion of the size of the lateral ventricles. Compared to the sham treatment group, the treated group had a 55% reduction of lateral ventricle area in standardized sections (FIG. 5). Foot clasping, a dystonic posturing of the hind limbs on suspension by the tail (FIG. 6a), is a characteristic behavior of the later stages of the disease in R6/2. CQ treatment decreased the average claspingscore at 10 weeks from 2.5 (of 3 maximum) to 1.3, a 48% reduction (FIG. 6b). The treated group also scored significantly better on rotarod performance, a test of agility and muscle coordination, between ninth and eleventh-weeks of age (FIG. 6c). The initial trend to a therapeutic effect of CQ on rotarod performance in R6/2 mice began when symptoms such as tremors and weight loss developed. With respect to weight changes, without CQtreatment, weight gain in R6/2 diverged from the wildtype curve and reached a plateau between the fifth and seventh week, which was followed by a steady weight loss (FIG. 6d). With CQ treatment, weight gain in R6/2 was comparable to that of the vehicle-treated group up to the seventh week of age but trended towards a gain thereafter, with weights significantly greater than untreated animals observed in the 10^{th} and 11^{th} weeks (FIG. 6d). The average lifespan of untreated animals in this series was approximately 76d, within the originally described range (10-13 weeks) of R6/2 lifespan (32). CQ treatment extended the lifespan to an average of 92 d, a 20% increase in longevity over the littermate control group (FIG. 6e). This falls within the range of survival improvement (7%-31%) seen with several therapeutic agents tested in this mouse model (reviewed in Beal et al, 2004 Supra).

### Example 13

CQ Mitigates HD of the Endocrine Pancreas

R6/2 mice develop diabetes mellitus due to dysregulation of insulin gene expression associated with sequestration of transcription factors by mutant Htt, which has suggested a mechanism for the elevated incidence of diabetes in individuals with HD (Andreassen et al, Neurobiol Dis 11:410-424, 2002; Hurlbert et al, Diabetes 48:649-651, 1999). We found that after 8 weeks of CQ treatment, 1′-week old R6/2 mice had normal fasting blood glucose levels (4.1±0.3 mM), comparable to that of wild-type littermate controls (5.2±0.8 mM). R6/2 sham treated animals had significantly higher fasting blood glucose levels (7.6±0.4), characteristic of diabetes (Table 3). Consistent with the blood glucose level was a reciprocal change in blood insulin levels. The sham group had a significantly lower fasting blood insulin level (166±5.13 μg/ml) than the treated group (415±54.60 μg/ml) (Table 3), which was similar to that of wild-type mice in these experiments and previously reported (Beal et al, 2004 Supra).

TABLE 3
Characteristic of diabetes

| | | Vehicle-treated | CQ-treated |
|---|---|---|---|
| Mice | Wild-type | R6/2 | R6/2 |
| Glucose level | 5.2 ± 0.8 | 7.6 ± 0.4 | 4.1 ± 0.3* |
| (mM) | | | |
| Insulin level | 403 ± 6.35 | 166 ± 5.13 | 415 ± 54.60** |
| (pg/ml) | | | |

Table 3 above shows results of fasting on plasma glucose and insulin levels in 11-week old mice. Data are mean+s.e.m. n≧4 for each treatment group. *P<0.0004 for glucose, **P=0.0065 for insulin levels comparing vehicle and CQ-treated groups.

### Example 14

Molecular Effects of CQ

The aims of this Example are four fold: a) to determine the effects of the metal chelators on expanded and normal HD protein production and toxicity in vitro; b) to determine whether CQ causes preferential truncation of polyQ expanded N-terminal Htt translation products; c) to determine the polyQ length threshold for differential CQ action in cell culture; and d) to determine whether variations in triplet coding or the sequence of coded translation products alter CQ effects.

CQ appears to selectively interfere with synthesis of a protein containing CAG repeats coding for polyglutamine. The elucidation of the mechanisms by which this occurs allows the isolation of the activities of CQ requisite for its positive effects on HD models, and lead to CQ modifications or new compounds with improved efficacy and less toxicity. The major known activity of CQ is to chelate metals, Zn and Cu in particular, and this action is considered to be important for its effects on Aβ in Alzheimer's disease (Bush, Trends Neurosci 26(4):207-214, 2003). Though chelation could have effects on DNA, RNA, protein synthesis and structure, including aggregation, reactive oxygen species generation, and other enzymatic activities, other functions independent of metal binding are possible. To affect translation, CQ could interact directly with RNA, with RNA binding proteins, with the ribosome, with the nascent translation product, or with other translation control mechanisms. To better understand the mechanisms of CQ action, a series of cell culture experiments are conducted directed at functional requirements of the drug, the nature of the translated product and the structural requirements of the mRNA.

In one experiment the question whether CQ mediated selective downregulation of polyQ-htt requires chelation is investigated. To examine the question of the role of chelation, the effects of several chemically dissimilar chelating agents is determined on polyQ-htt synthesis. Since none of the compounds will precisely emulate the chelation characteristics and cellular distribution of CQ, a broad range of compounds is examined. These include: the cell-permeable chelators neocuproine (2,9-dimethyl-1,10-phenanthroline), Zinquin ((2-methyl-8-p-toluenesulphonamido-6-quinolyloxy)acetic acid), diethyldithiocarmabate, 1,10-phenanthroline, the non-permeable chelators bathocuproinedisulfonic acid and picolinic acid (pyridine-2-carboxylic acid); and N-acetylcysteine amide which is known to cross the blood brain barrier. These compounds all bind zinc, copper and iron ions, though with differing affinities. Most bind copper several orders of magnitude more tightly than zinc, though for Zinquin the difference is less than one order of magnitude. Most are expected to be toxic at higher concentrations. The reproduction of the CQ effect by another chemically distinct chelator implicates chelation of a metal ion.

In another experiment, it is determined whether the translated product is truncated by the action of CQ. The expanded polyQ mRNA transcripts are proposed to imitate translation normally in the presence of CQ, but ware interrupted or stalled at or before the expanded repeat segment, releasing one or several truncated products, which may be rapidly metabolized. After proteasomal degradation, smaller peptides are metabolized principally by cytoplasmic aminopeptidases and in some cases by thimet oligopeptidase. Such stalling has been observed, due to extensive stable RNA secondary structure formation. By immunotagging the N-terminus of the construct, collecting the translated products and analyzing them by mass spectrometry, it is possible to follow the translation process and identify stalling or truncation points.

In a third experiment, the question of the CAG length dependence of the CQ effect is examined. It is proposed that the CQ effect involves an interaction between the expanded CAG repeat mRNA and the translation apparatus. This process will not necessarily reflect the length dependence of polyQ toxicity, which, in the context of huntingtin, begins at 36-38 repeats. Thus, it could be that individuals with shorter alleles in the disease-producing range (>38), might not respond to CQ, or individuals with longer CAG segments in their non-toxic allele might shut down all huntingtin expression. Determining the length dependence of the CQ effect provides guidance in the application of CQ with respect to repeat length, as well as providing further mechanistic clues.

A fourth experiment characterizes the structural requirements for a transcript to respond to CQ. If mRNA secondary structure, in particular hairpinning, is important, but not the protein coded, then small changes in the RNA sequence that interrupt the polyglutamine segment with minimal changes in the secondary structure (e.g. introduction of proline), should still respond. Similarly, eliminating the potential for RNA hairpinning while maintaining the polyglutamine coding (CAA repeats) should yield a transcript that does not respond. Finally, a GUC repeat (polyval, which should hairpin and form aggregates) should respond to CQ, while polylysine repeats (polyAAA/AAG, which should not hairpin, but are known to form nuclear aggregates), should not.

PC12 cells are cultured and transfected as described above. All compounds noted above are available from commercial sources. Cells cultured in Optimem media in 24-well plates are pretreated 30 minutes prior to transfection with each of the compounds in DMSO, initially at concentrations of 0.05, 0.1, 0.5, 1, 2, 5 and 10 μM, or vehicle alone and transfected, using Lipofectamine 2000, with Q55HDexonI/GFP, Q15HDexonI or GFP alone vectors. Cells are then digitally imaged (Nikon inverted fluorescence microscope with Magnafire digital imager) under phase contrast and for GFP fluorescence at ˜20 and 40 hours post transfection. At 48 hours they are stained with propidium iodide (PI) and Hoechst 33342 and imaged again with filters appropriate for those dyes. The percentage of cells exhibiting detectable GFP fluorescence or PI staining is determined by semi-automated counting (using NIH image) of the same field under fluorescent and phase imaging. Apoptotic nuclear fragmentation of PI negative cells is assessed by manual visual counting using overlapped PI/Hoechst images. This, plus PI positive cells will be considered to represent the total number of dead cells. At least 3 fields will be counted per condition, and the experiment repeated at least 4 times after the initial concentration screen. Should a compound prove toxic or ineffective but not toxic in the initial concentration range, the range is extended accordingly. Statistical significance for between group comparisons is determined by ANOVA, with differences between individual values determined by post hoc testing with Dunnet's test. Any compound and concentration that produces a substantial (>25%) drop in percentages of Q55 expressing cells, with at most a 3-fold lesser drop in Q15 and GFP-only expression, is considered for further evaluation, including analysis of mRNA expression by Northern blotting or reverse transcription quantitative PCR, Western blotting and densitometric quantitation with anti-polyQ and antiGFP, and proteasome inhibitor treatment.

Using standard cloning methods, the ‘FLAG’ immunotag sequence will be introduced into the N-terminus of the currently used Q55 and Q15HDexonI constructs, so that the expressed peptide will contain the sequences

MDYKKDDDDKATLEKLMKAFESLKSF-(Q)ₙ—PPPPPPPPPPPQLPQPPPQAQPLLPQPQPPPPPPPPPPGPAVAEEPLHRP-GFP (flag underlined; HDexonI, italics; green fluorescent protein, bold, SEQ ID NO:2). PC12 cells grown in 100 mm dishes is treated with 2 or 4 μM CQ and transfected with the FLAGQ55 or FLAGQ15 constructs, or the unFLAGed constructs as control. Six hours after transfection, cultures are treated or not with the proteasome inhibitor MG 132, the aminopeptidase inhibitor bestatin (Sigma), and/or the thimet oligopeptidase inhibitor Cpp-AAF-pAb (Calbiochem-Novabiochem, San Diego, Calif.). 3, 6 and 12 hours later, cell extracts are immunoprecipitated with anti-FLAG and, using standard protocols, captured on protein-G derivatized SELDI (surface-enhanced laser desorption ionization) chips for Mass Spectrometric display on a Ciphergen PBSII MALDI-TOF (matrix-assisted laser desorption ionization-time of flight) mass spectrometer. In the mass range of the expected peptides, this device has a mass accuracy of a few Daltons, usually making possible mapping/identification of a known sequence.

Beginning with the Q15HDexonI-GFP construct, using standard PCR and oligonucleotide cloning techniques, a series of expanded CAG repeat vectors is made, cloned and sequenced, and clones selected representing a sequence of repeat lengths including 20, 25, 30, 35, 38, 40 and 45 repeats. They are resequenced at intervals to ensure the fidelity of the repeat number. These vectors are transfected into CQ treated and untreated PC12 cells and compared with Q15-GFP and GFP-alone as described above.

Beginning with the vectors in hand, the following sequences are constructed encoding: (Q₂₄-P-Q₃₀)HDexonI-GFP, Q55HDexonI with the Qs encoded entirely by CAA, V55 and V15HDexonI-GFP with polyv encoded by GTC repeats, and K55 and K15HDexonI-GFP with polyK encoded by AAG repeats. These vectors are transfected into PC12 and evaluated for CQ effect as above.

Some of the chelating agents are likely be toxic and cause a global non-selective decrease in protein synthesis. There is a reasonable chance that none will reproduce the effects of CQ, suggesting that the actions of interest are not due to chelation alone. It would remain possible that none of the selected compounds is sufficiently similar to CQ with respect to binding affinities, cellular distribution or some other factor. This could prompt further studies of available or synthesized congeners of clioquinol, with and without chelating properties, to further define structure-activity relationships, and shift the emphasis away from a metal intermediary in the mechanism of action. It might also be possible to develop a cell-free system that reproduces the effect and allows more control of the ionic environment. It is expected that CQ will cause progressive ribosomal stalling and the formation of short translation products which may or may not be released from the ribosome. The total cell extracts of the initial studies would not be expected to distinguish between these possibilities. In either case the products truncated at a site in the vicinity of the upstream end of the CAG repeat are expected to be identifiable by mass spectrometry. They may only be observable, however, with inhibition of the proteasome and/or cytosolic proteases, depending on whether they are released and their length. Longer products could be depleted with anti-GFP prior to display, to enhance short product detection. If no short products are observed it may be that CQ affects translation far enough upstream as to interfere with the immunotag, or perhaps at initiation, which could prompt studies of initiation factor recruitment and ribosomal engagement. It is possible the FLAG sequence itself could interfere with the effect, and if the new construct did not respond to CQ as expected, another small N-terminal immunotag such as HA or myc could be used. By studying the fate of full length translation products over a longer period of time, in the presence or absence of selective protease inhibitors the effect of degradation can be assessed. It is anticipated that the effect of CQ will occur within the specified length range, though it may be above or below the transition to disease-producing lengths. If none of the constructs in the specified range respond, the range may be extended. It is expected that the effect of CQ will correlate with the ability of the RNA to form hairpin secondary structures rather than protein-related parameters, and so Q₂₄-P-Q₃₀ and polyv constructs should respond, while polyQ(CAA) and polyK do not. If Q₂₄-P-Q₃₀ aggregates significantly, Q₁₅-P-Q₁₅-P-Q₂₃ or the equivalent is tried.

### Example 15

Dose Response for Oral CQ

This experiment determines a dose-response for oral CQ treatment of transgenic HD (R6/2) mice and determines the effects of longer term therapy in a less severe HD model (YAC72).

Lower dosages need to be investigated to determine if such doses improve the effect. It is also important to examine a longer term, milder model, which perhaps better emulates the course of the disease in humans, and can demonstrate sustained effectiveness of the therapy. Moreover, since the drug is given as a single daily dose in a water suspension, it is possible that the window of time in which the blood levels are therapeutic may be small, perhaps only a few hours a day. To address these issues, it is proposed to use the R6/2 short term model for a dose-response study and then examine the effects of treatment on the longer term, milder YAC72 model. YAC72 animals carry a portion of a yeast artificial chromosome (YAC) bearing a full-length human htt gene (including the promoter) that is modified with a 72 CAG repeat expansion in exon 1. These mice exhibit altered hippocampal long-term potentiation by 6 mos and accumulation of Htt fragments, and striatal neurodegeneration by 9 mos of age. There are few behavioral abnormalities, though they may exhibit clasping, circling behavior and hyperactivity, and their lifespan is not shortened. In order to distribute the dosage in time and make longer term treatment feasible, the dosage is delivered in food rather than by water gavage.

Animals are bred and genotyped with the addition of a strategy to minimize the tendency of CAG repeat length to lengthen, especially when transmitted by males. This involves sequencing the genotyping PCR products to determine CAG repeat length prior to breeding and only breeding animals at the lower end of the length distribution. In addition, weight data are compared by sex and adjusted for weight at weaning, ensuring a standardized environment, randomizing based on litter origin, and housing animals with the equal numbers of wild type and transgenics in each cage.

CQ is delivered blended into food pellets with vitamin B₁₂ supplementation. In initial brief studies with wild-type adult animals, it is ensured that the introduction of CQ into the diet does not make the food unpalatable (prepared by Research Diets, Inc, New Brunswick, N.J., USA), by comparing consumption rates by (by weighing of food) between undosed and CQ containing food at each dosage level. In addition, it is determined what is the average rate of consumption and fluctuations in consumption rate through the course of the day. These parameters are measured periodically in test subjects as well, to allow adjustment of dosing for body weight and food intake. In the first set of experiments, groups of R6/2 and are treated from weaning at average daily dosages of 0, 15, 30, 45 and 60 mg/kg/day. Power analysis suggests 18 animals per group for behavioral studies and survival (using clasping as a surrogate, SD_{1,2}=1.1, P=0.05, power=0.9, μ₁=2.5, μ_{2=1.3}) and 5 animals per group for pathologic studies (using ventricle area as a surrogate, SD₁=0.098, SD₂=0.18, P=0.05, power=0.9, μ₁=0.265, μ₂=0.59). Given the possibility of loss of animals in the pathology group prior to 12 week harvesting, 2 animals are added per group, for a total of 25 animals/group. An untreated wild-type control group is included as well. Animals are evaluated for a variety of parameters including clasping, rotarod performance, body weight, and survival, and animals are sacrificed at 12 weeks for western and immunohistochemical analysis of huntingtin fragment accumulation and striatal atrophy. Animals that decline more rapidly than control in any of these parameters are considered to be experiencing drug toxicity. The optimal dose, based on maximal efficacy and minimal toxicity, will then be applied to the YAC72 model. Assuming similar statistical requirements, groups of 18 (ongoing behavioral studies)+5×3 (timepoints)=33 animals each of YAC72 with and without treatment, and wildtype littermates will be followed for up to 18 mos, at 3 month intervals, for the development of clasping and videotaped and scored (frequency/2 hrs) for a variety of behaviors, including: inactivity, locomotion, scratching, sniffing, jumping, gnawing, burying, rearing, circling, and grooming. 5 from each group will be sacrificed at 9, 12, and 18 months for polyQ westerns and immunohistopathology (polyQ staining with EM48) and visual counting of neurodegeneration in thin sections of striatum (the only part of the brain to show neurodegeneration in this model), stained with toluidine blue, with neuronal nuclear profiles scored as shrunken & hyperchromatic, showing morphologic abnormalities, and normal. Comparisons between groups are made by ANOVA.

It is expected that the R6/2 animals will show a graded dose response of all parameters to CQ treatment and that the response curve will plateau at higher dosages. The changes in delivery incurred by the delivery in food may provide a more sustained blood level, but might also be inadequate at all dosages proposed, due to the need for a high peak level, or poorer absorption. For this reason, higher dose regimens are evaluated early, to allow for subsequent adjustments. It is expected that chronic CQ administration with B₁₂ supplementation will be well tolerated, and significantly mitigate the behavioral and pathologic features of YAC72 animals. If the animals appear not to tolerate long term therapy at the dosage suggested by the short term experiments, the dose can be decreased. If they appear not to be responding with respect to behavior and pathology at the first time points, a dose increase is considered. Finally, if they do not respond, it may be due to differences in that model in htt transcript synthesis, processing or translation, since they express the full length gene rather than exon I only (as in R6/2), and have fewer repeats.

### Example 16

CQ Mitigates Toxic Product Production

This Example aims to determine whether CQ mitigates toxic product production and cell death in in vitro models of spinocerebellar atrophy (SCA) types 1 and 3, and Kennedy's disease, and in a mouse model of SCA2.

If the effects of CQ are likely due to interactions with the CAG translation process, then other diseases characterized by CAG expansions may also respond to this therapy. As noted above, other polyglutamine-expansion diseases display varying manifestations that depend upon the gene in which the expansion resides. Overall, they are rare diseases, though SCA-1, 2 and 3 are the most prevalent of the hereditary ataxias, and Kennedy's disease may be a leading diagnostic alternative to ALS in some populations. They are due to expanded repeats in, for the SCAs, ataxin-1, ataxin-2 and ataxin-3 (also known as the Machado-Joseph or mjd protein), and for Kennedy's disease, the androgen receptor (AR). The polyQ region resides in the N-terminal quarter of ataxin 1 & 2, and AR, while it is in the C-terminus of ataxin-3. Expression of polyQ-expanded ataxin-1, 3 and AR causes aggregate formation and cell death in cell culture models. The transgenic SCA-2 mice (B6D2-Tg(Pcp2SCA2)11Plt/J) express human SCA2 containing 58 repeats under the direction of the mouse Pcp2 promoter. Visualization of the protein in the cerebellum with anti-SCA2 or anti-polyglutamine antibody (IC2) shows it is highly expressed in the cytoplasm and does not form nuclear aggregates. A loss of calbindin-28K positive Purkinje cells begins at 4 weeks with a progression to 50-60% loss by 26 weeks in heterozygotes. Progressive functional deficits occur as early as eight weeks of age as measured by hind leg clasping and stride length, and rotarod testing.

Full length normal and polyQ-expanded ataxin 1 and 3, and truncated AR cDNAs are obtained. Using standard procedures, they are fused to eGFP and transfected into PC12 cells, treated with a concentration range of CQ and evaluated for protein product accumulation and cell death with comparisons of percent cells producing detectable fluorescence, percent cell death and western blotting of polyQ accumulation.

SCA2 heterozygous mice are obtained from Jackson laboratories. Animals are produced for experiments by backcrossing against the parent C57BI/DBA strain and genotyped using PCR. Assuming statistical considerations and since they are not expected to reach mortality within the 28 week time course, groups of 18 animals are treated with 0 or 30 mg/kg/day (or other dose determined to be optimal) in their food, and are evaluated at 6 week intervals for clasping and rotarod performance (4 consecutive day trials). At 28 weeks 5 animals in each group are sacrificed and brains recovered to evaluate numbers of Calbindin-28K, 1C₂ anti-polyglutamine antibody (Chemicon) and ataxin-2 (Zymed) staining Purkinje neurons in the cerebellum and western blotting for these as well. Statistical comparisons are by ANOVA as before.

It is expected that CQ will decrease the accumulation of the mutant, toxic form of the protein, without affecting the normal form, in each of the cell culture models. If it does not, it may indicate a context dependence of the mechanism, perhaps due to differences in RNA secondary structure, RNA protein binding or interactions of the N-terminus of peptide product while in translation. If there is a contribution of differential degradation, this would likely also be different for different protein products. Similarly, it is expected that CQ treatment will decrease the progression of behavioral abnormalities, the accumulation of ataxin-2 in and loss of Purkinje cells in the cerebella of SCA2 mice.

### Example 17

High Throughput Screening

This Example aims to develop and use a dual reporter protein containing a C-terminal green and N-terminal red fluorescent protein and an intervening polyQ segment, to perform high throughput screening for new compounds that selectively inhibit transcription or translation of expanded CAG repeats.

Though CQ may hold clinical promise for HD and related diseases, it may ultimately prove ineffective due either to fundamental mechanistic differences between the animal test models and humans and/or issues of toxicity. Thus there is a need to continue to identify new small drugs or drug-like compounds that may treat these diseases with nominal toxicity. Several groups have approached this problem with high throughput assay paradigms are proposed focused on aggregation or cell survival. In one approach, an automated in vitro filter retardation assay to identifies compounds that inhibit HDexon1 protein aggregation. In another approach, a fluorescence resonance energy transfer (FRET)-based assay issued to assess intracellular aggregation of polyQ-expanded androgen receptor fragment-fluorescent protein fusions. Another approach uses using an in vitro assay based on measuring turbidity of a sperm whale myoglobin with an incorporated 35Q segment and cell-based assay of Q60htt-eGFP aggregate formation.

CQ may selectively interfere with expanded CAG-repeat translation. Hence, it is proposed to use a screening paradigm to more efficiently identify new compounds that function at the earliest stages of the disease process. This will potentially result in more effective and perhaps selective actions, affecting both cell dysfunction and death, compatible with long term administration and available in a shorter time-frame than other therapies (e.g. DNA-based, RNAi, transplantation). The assay involves the expression of a dual color N(red) and C(green)-terminal fluorescent protein labeled reporter that, in the case where translation is interrupted by the action of a compound, yield only the red color, changing the red/green (R/G) ratio of the signal. Such dual vectors have been successfully used to measure protease activity. If a compound significantly increase the R/G ratio in cells expressing the expanded Q form relative to unexpanded, it is a candidate selective inhibitor of translation. Moreover, if the R^{Q55}/R^{Q15} ratio were diminished by a drug, there might be a selective transcriptional, transcript processing, or early translational action.

A vector expressing dsRED red fluorescent protein (RFP) (BD Biosciences) is used and it is inserted this at the 5′ end of the Q15HDexonI-eGFP construct to create RFP-Q15HDexonI-eGFP (RQ15G). A RFP-Q55HDexonI-eGFP(RQ55G) vector is also constructed. In preliminary experiments, HEK293 cells are transfected and by fluorescence microscopy confirm that RQ55G aggregates and causes cellular toxicity by 48 hrs as Q55G does, and that RQ15G does not. HEK293 cells grown in 100 mm dishes are transfected with RQ55G or RQ15G using standard protocols, and 6 hours later they are harvested by trypsinization and plated in 96 well plates at 20,000 cells/well. Using a fluorescent plate reader green (G, Ex 485, Em 520), red (R, Ex 544, Em 604) and FRET/bleedthrough (Ex 485, Em 604) are measured signals determined. FRET occurs when two fluorophores are close proximity (<100 angstroms) and the emission spectrum of one overlaps with the excitation spectrum of the other, allowing direct energy transfer from the acceptor to the donor. The result is that the signal from the donor is diminished while that of the acceptor is augmented. Bleedthrough is signal in channels other than the intended color occurring due to overlap of excitation spectra, e.g. RFP has significant absorbance at the GFP excitation wavelength 485. In this case bleedthrough can be assessed by determining FRET channel signal relative to GFP signal in cells co-expressing unconjugated RFP and GFP (which do not exhibit FRET). It is expected that the constructs will exhibit considerable FRET as is the case for similar constructs. In addition, there may be additional FRET with the RQ55G products due to self association. In practice, if a compound causes product truncation such that only the RFP is made, then R/G should increase due to a change in the ratio of protein concentrations. Since there should be no significant FRET due to red excitation in this system, the absence of GFP should not affect the RFP signal. The system is tested and validated by analysis of cells co-expressing the full length RQ55G reporter with varying amounts of RFP alone. Correlation of the ratio of R to RQ55G, which may be further determined by western blotting, provides an estimate of the sensitivity of the assay. For high-throughput screening, 2 hrs after plating compounds from the ActiProbe-10 (Timtec) collection of 10,000 diverse, drug-like molecules are added to final concentrations of 0 (vehicle alone), 0.05, 0.5 or 5 μg/ml (which should represent between 0.1-0.3, 1-3 and 10-30 μM for most of the compounds in the collection with MWs of 150-500 D), in triplicate to wells containing cells transfected with either RQ55G or RQ15G. Thus, each compound occupy 24 wells allowing assessment of 4 compounds/plate. Initial comparisons include (R^{Q55})_{compound} to (R^{Q55})_{vehicle} and (R^{Q15})_{compound} to (R^{Q15})_{vehicle} to determine whether the compound whether the compound reduces red signal and possibly transcription in a global or specific fashion and (R/G-Q55)_{compound}/(R/G-Q55)_{vehicle} to (R/G-Q15)_{compound}/(R/G-Q15)_{vehicle} to determine whether it causes an excess of red signal, possibly due to a specific interruption of translation. Statistical evaluation of the comparisons is done by ANOVA with post-hoc Dunnet's testing. Compounds showing significant apparent selective activity are further evaluated with fluorescence microscopy, analysis of toxicity (PI and Hoechst staining), protease inhibitor treatments, quantitative PCR or Northern blotting, and western blotting.

It is expected that this screening paradigm will yield compounds that differentially down-regulate expanded polyQ protein production. Though there are numerous potential mechanisms for such an effect, for example, differential degradation of expanded RNA, or acceleration of polyQ degradation, it is expected that some of the compounds will obtain a mechanism like that suggested for CQ, i.e. preferential interruption of translation at the CAG repeat. It is expected that numerous compounds will be toxic, and some may show differential toxicity, augmented by the expanded polyQ expression, emulating a differential reduction in polyQ synthesis. These will be identified in second round toxicity analysis. If none of the compounds is positive in the primary screen, those that are toxic at the lowest concentration are rescreened at lower concentrations, while those that were ineffective, but not toxic at the highest concentration are rescreened at higher concentrations. The application of the requirement for specificity may engender the need to screen more compounds to find a fewer candidate drugs than in other studies. If R/G is observed to decrease, it could be due to inactivation or destruction of the N-terminal RFP, resulting in altered concentration ratios of fluorophores and loss of FRET, or cleavage between the GFP and RFP moieties, which while retaining concentration parity, would also result in loss of FRET, increasing the signal from GFP. Comparisons of relative RFP signals and FRET measurements should aid in the interpretation. Finally, compounds which are the most effective, selective and least toxic through secondary and tertiary in vitro analysis will be considered for animal trials.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### IV. BIBLIOGRAPHY

Ambrose et al, Somat Cell Mol Genet. 20:27-38, 1994
Andreassen et al, Neurobiol Dis 11:410-424, 2002
Arrasate et al, Nature 431:805-810, 2004
Bates et al, Huntington's Disease (Oxford University Press, Oxford), 2002
Beal M F & Ferrante, Nat Rev Neurosci 5:373-384, 2004
Bush, Trends Neurosci 26(4):207-214, 2003
Choo et al, Hum Mol Genet. 13:1407-1420, 2004
Davies et al, Cell 90:537-548, 1997
Dunah et al, Science 296:2238-2243, 2002
Gafni et al, J Biol Chem 279:20211-20220, 2004
Gauthier et al, Cell 118:127-138, 2004
Goodman and Gilman's “The Pharmacological Basis for Therapeutics”, 7^{th} Ed., 1985
Gunawardena et al, Neuron 40:25-40, 2003
Heiser et al, Proc Natl Acad Sci USA 99(4):16400-16406, 2002
Hockly et al, Brain Res Bull 61, 469-479, 2003
Holmberg et al, Embo J 23:4307-4318, 2004
Hurlbert et al, Diabetes 48:649-651, 1999
Jiang et al, Hum Mol Genet. 12:1-12, 2003
Meade et al, J Comp Neurol 449:241-269, 2002
Petersen et al, Neurology 56:1133-1142, 2001
Pollitt et al, Neuron 40:685-694, 2003
Remington's Pharmaceutical Sciences, 20^{th} Edition, Williams & Wilkins, Pennsylvania, USA
Ritchie et al, Arch Neurol 60:1685-1691, 2003
Ruan et al, Hum Mol Genet. 13:669-681, 2004
Schaffar et al, Mol Cell 15:95-105, 2004
Schilling et al, Hum Mol Genet. 8:397-407, 1999
Snell et al, Nat Genet. 4:393-397, 1993
Staber et al, Huntingtin Gene Silencing by Lentivirus-Delivered SHRNA Leads to Phenotypic Improvement in an HD Mouse Model Program No. 1015.2, 2004 Abstract Viewer/Itinerary Planner, Washington, D.C. Society for Nueroscience, 2004
Storey et al, Ann Neurol 32:526-534, 1992 The British National Formulary 43^{rd} Ed. (British Medical Association and Royal Pharmaceutical Society of Great Britain, 2002; <http://bnf.rhn.net>)
Watase et al, Neuron 34:905-919, 2002
Xia et al, Nat Med 10:816-820, 2004
Yamamoto et al, Cell 101:57-66, 2000
Young A B, J Clin Invest 111:299-302, 2003
Zuccato et al, Science 293:493-398, 2001
Zuccato et al, Nat Genet. 35:76-83, 2003

What is claimed is:

1. A method for the treatment of a polyglutamine (polyQ)-expansion disease in a subject in need of such treatment, said method comprising administering to said subject an effective amount of an 8-hydroxyquinoline compound having the formula 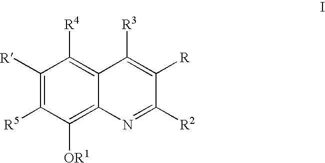
wherein,
R, R′, R², and R³ are H;
R¹ is H or unsubstituted C₁-C₁₀ alkyl; and
R⁴ and R⁵ are independently halogen or unsubstituted C₁-C₁₀ alkyl.

2. The method of claim 1 wherein R¹ is H or unsubstituted C₁-C₆ alkyl.

3. The method of claim 1 wherein R¹ is H or unsubstituted C₁-C₃ alkyl.

4. The method of claim 1 wherein R¹ is H or methyl.

5. The method of claim 1 wherein R¹ is H.

6. The method of claim 1 wherein R⁴ and R⁵ are independently halogen or unsubstituted C₁-C₆ alkyl.

7. The method of claim 1 wherein R⁴ and R⁵ are independently halogen or unsubstituted C₁-C₃ alkyl.

8. The method of claim 1 wherein R⁴ and R⁵ are independently halogen or methyl.

9. The method of claim 1 wherein R⁴ and R⁵ are independently halogen.

10. The method of claim 1 wherein R⁴ and R⁵ are independently Cl or I.

11. The method of claim 1 wherein R¹ is H; and R⁴ and R⁵ are independently halogen.

12. The method of claim 1 wherein the 8-hydroxyquinoline compound is clioquinol.

13. The method of claim 1 wherein the polyQ-expansion disease is Huntington's disease (HD).

14. The method of claim 1 wherein the polyQ-expansion disease is selected from the list consisting of spinal and bulbar muscular atrophy, dentatorubral-pallidoluysian atrophy, spinocerebellar ataxia (SCA) types 1, 2, 3(Machado-Joseph Disease), 7 and 12.

15. The method of claim 1 wherein the subject is a human.

16. The method of claim 1 wherein the subject is an animal selected from dogs, cats, mice, rats, horses, ponies, donkeys, mules, llama, alpaca, pigs, cattle, sheep, primates, felids, canids, bovids or ungulates.

17. The method of claim 1 further comprising the simultaneous or sequential administration of one or more of a histone deacetylase inhibitor, a caspase inhibitor, a mitochondrial/energy modulator, an antiexcitotoxicity agent, an anti-aggregation agent, RNAi, an immunointeractive molecule and/or a small molecule inhibitor of polyQ-Htt.

18. The method of claim 1 wherein the 8-hydroxyquinoline compound is administered from about 100 to about 1500 mg/day.

19. A method for ameliorating the symptoms of HD or other polyQ-expansion disease in a subject, said method comprising administering to said subject an effective amount of an 8-hydroxyquinoline compound having the formula 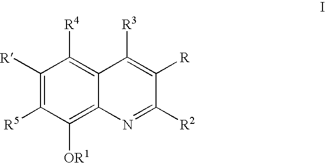
wherein,
R, R′, R², and R³ are H;
R¹ is H or unsubstituted C₁-C₁₀ alkyl; and
R⁴ and R⁵ are independently halogen or unsubstituted C₁-C₁₀ alkyl.

20. The method of claim 19 wherein the polyQ-expansion disease is selected from the list consisting of spinal and bulbar muscular atrophy, dentatorubral—pallidoluysian atrophy, spinocerebellar ataxia (SCA) types 1, 2, 3(Machado-Joseph Disease), 7 and 12.

21. The method of claim 19 wherein the subject is a human.

22. The method of claim 8 or 9 wherein the subject is an animal model selected from the list consisting of dogs, cats, mice, rats, horses, ponies, donkeys, mules, llama, alpaca, pigs, cattle, sheep, primates, felids, canids, bovids and ungulates.

23. The method of claim 19 further comprising the simultaneous or sequential administration of one or more of a histone deacetylase inhibitor, a caspase inhibitor, a mitochondrial/energy modulator, an antiexcitotoxicity agent, an anti-aggregation agent, RNAi, an immunointeractive molecule and/or a small molecule inhibitor of polyQ-Htt.

24. The method of claim 19 wherein the 8-hydroxyquinoline compound is administered from about 100 to about 1500 mg/day.

25. A pharmaceutical composition comprising one or more of a histone deacetylase inhibitor, a caspase inhibitor, a mitochondrial/energy modulator, an antiexcitotoxicity agent, an anti-aggregation agent, RNAi, an immunointeractive molecule and/or a small molecule inhibitor of polyQ-Htt; one or more pharmaceutically acceptable carriers and/or diluents; and an 8-hydroxyquinoline compound having the formula 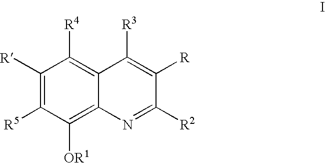
wherein,
R, R′, R², and R³ are H;
R¹ is H or unsubstituted C₁-C₁₀ alkyl; and
R⁴ and R⁵ are independently halogen or unsubstituted C₁-C₁₀ alkyl.


SEQUENCE LISTING (3 sequences)

SEQ ID NO: 1 - DNA, 18 bp, Artificial Sequence
  Description of Artificial Sequence Synthetic oligonucleotide
caacagcaacaacagcag

SEQ ID NO: 2 - protein, 77 aa, Artificial Sequence
  Description of Artificial Sequence Synthetic peptide construct
MDYKKDDDDKATLEKLMKAFESLKSFQPPPPPPPPPPPQLPQPPPQAQPLLPQPQPPPPPPPPPPGPAVAEEPLHRP

SEQ ID NO: 3 - protein, 76 aa, Homo sapiens
MDYKKDDDDKATLEKLMKAFESLKSFPPPPPPPPPPPQLPQPPPQAQPLLPQPQPPPPPPPPPPGPAVAEEPLHRP